(19) United States
(12) Patent Application Publication
(10) Pub. No.: US 2014/0243218 A1
(43) Pub. Date: Aug. 28, 2014

(54) COMPOSITION FOR DIAGNOSING OVARIAN CANCER METASTASIS USING CPG METHYLATION STATUS OF GENE PROMOTER AND USE THEREOF

(71) Applicant: Ewha University - Industry Collaboration Foundation, Seoul (KR)

(72) Inventors: Jung-Hyuck AHN, Seoul (KR); Hye-Youn Sung, Seoul (KR); Woong Ju, Seoul (KR)

(73) Assignee: Ewha University - Industry Collaboration Foundation, Seoul (KR)

(21) Appl. No.: 14/185,099

(22) Filed: Feb. 20, 2014

(30) Foreign Application Priority Data
Feb. 28, 2013 (KR) 10-2013-0022240
Feb. 28, 2013 (KR) 10-2013-0022243
Feb. 28, 2013 (KR) 10-2013-0022244
Feb. 28, 2013 (KR) 10-2013-0022245
Feb. 28, 2013 (KR) 10-2013-0022246

(51) Int. Cl. C12Q 1/68

(52) U.S. Cl. CPC C12Q 1/6886; C12Q 2600/154; C12Q 2600/118; C12Q 2600/112
USPC 506/7

(57) ABSTRACT

The present invention relates to a composition, a kit and a method for diagnosing ovarian cancer metastasis or predicting the risk of metastasis by detecting methylation levels at CpG sites of one or more gene promoters selected from the group consisting of AGR2 (anterior gradient 2), CA9 (carbonic adj anhydrase 9), GABRP (gamma-aminobutyric acid receptor pi subunit), IFITM1 (interferon-induced transmembrane 1) and MUC13 (mucin 13).

### CROSS REFERENCE TO RELATED APPLICATION

This application claims priority to and the benefit of Korean Patent Application No. 10-2013-0022240 filed on Feb. 28, 2013, Korean Patent Application No. 10-2013-0022243 filed on Feb. 28, 2013, Korean Patent Application No. 10-2013-0022244 filed on Feb. 28, 2013, Korean Patent Application No. 10-2013-0022245 filed on Feb. 28, 2013, and Korean Patent Application No. 10-2013-0022246 filed on Feb. 28, 2013, which are hereby incorporated by reference for all purposes as if fully set forth herein.

### BACKGROUND OF THE INVENTION

(a) Field of the Invention

The present invention relates to a composition, a kit and a method for diagnosing ovarian cancer metastasis or predicting the risk of metastasis by detecting methylation levels at CpG sites of one or more gene promoters selected from the group consisting of AGR2 (anterior gradient 2), CA9 (carbonic adj anhydrase 9), GABRP (gamma-aminobutyric acid receptor pi subunit), IFITM1 (interferon-induced transmembrane 1) and MUC13 (mucin 13).

(b) Description of the Related Art

Ovarian cancer is an intractable cancer having the highest mortality rate of female cancers, and the incidence continues to increase with westernized lifestyle, hormone replacement therapy and increasing aged populations. There are no distinct symptoms of early ovarian cancer. It has been reported that more than about 70% of patients are diagnosed with advanced ovarian cancer at stage 3 or greater and more than about 75% of patients experience a recurrence or metastasis within the first 2 years after initial treatment.

The treatment for ovarian cancer depends on the type and stage of cancer, which includes surgery, radiation therapy, chemotherapy or the like. These treatment methods show no great therapeutic effects on metastatic cancer from recurrence, because cancer metastasis is accompanied by angiogenesis and cell migration and is a different process from cancer itself. Thus, angiogenesis and cell migration should be also prevented in order to prevent cancer metastasis, because anti-metastatic and anticancer actions are different from each other. Accordingly, diagnosis of cancer is important, but development of biomarkers for predicting recurrence and metastasis after treatment of ovarian cancer is expected to greatly contribute to improvement of survival rate and treatment efficiency. Further, prediction of cancer recurrence and metastasis requires development of biomarkers that are different from the cancer diagnostic biomarkers, because there is an underlying difference between cancer metastasis or recurrence and tumorigenesis.

In more detail, it has been reported that metastatic cancer has biological characteristics different from those of the primary cancer, because there are differences in gene expression patterns between metastatic cancer and primary cancer. For instance, various growth hormones are needed for tumor cell growth, and changes in gene expression favorable to survival of metastatic cancer cells are ultimately required because metastatic cancer cells must overcome the anticancer effects to survive. It seems that these expression patterns play a very important role in determining the cancer metastasis. Therefore, it is hard to impute a cause of metastasis to a high expression level of a single gene of the related genes in tumor cells (primary site).

On the other hand, Korean Patent NO. 0983386, Japanese Patent Publication NOs. 2008-520228 and 2009-505632, Korean Patent NO. 1007571, and Korean Patent Publication NO. 2012-0034593 disclose that the genes selected in the present invention can be used as diagnostic markers for various cancers. However, the present invention clearly differs from these documents in that recurrence and metastasis of ovarian cancer are diagnosed or predicted using site-specific hypomethylation at CpG sites in promoters of the corresponding genes.

### SUMMARY OF THE INVENTION

In the present invention, gene expression patterns between primary cancer cells and metastatic tissues were compared. Of the genes showing changes in their expression patterns in the metastatic tissues, genes with CpG methylation changes in the promoter region were finally selected. Furthermore, the specific CpG sites affecting the gene expressions were identified, and methylation levels at the specific CpG sites of the corresponding gene promoters were measured so as to predict the risk of ovarian cancer metastasis, leading to the present invention.

An object of the present invention is to provide a composition for diagnosing ovarian cancer metastasis or predicting the risk of metastasis, including an agent measuring methylation levels at the CpG sites of one or more gene promoters selected from the group consisting of AGR2 (anterior gradient 2), CA9 (carbonic adj anhydrase 9), GABRP (gamma-aminobutyric acid receptor pi subunit), IFITM1 (interferon-induced transmembrane 1) and MUC13 (mucin 13).

Another object of the present invention is to provide a kit for diagnosing ovarian cancer metastasis or predicting the risk of metastasis, including the composition.

Still another object of the present invention is to provide a method for diagnosing ovarian cancer metastasis or the risk of metastasis, including the steps of:

(a) measuring methylation levels at the CpG sites of one or more gene promoters selected from the group consisting of AGR2, CA9, GABRP, IFITM1 and MUC13 in a biological sample of a subject,

(b) comparing the methylation levels with those of the gene promoters of a control sample, and

(c) determining that the subject has ovarian cancer metastasis or is at the risk of the metastasis when the methylation levels measured in the sample of the subject are lower than those of the control sample.

According to the present invention, methylation levels in the specific gene promoter regions of genomic DNA collected from a biological sample of a patient are measured by MSP (methylation-specific PCR) so as to diagnose the risk of ovarian cancer metastasis within several hours, thereby developing an accurate and convenient diagnostic kit.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

FIG. 1 is a diagram showing integration of mRNA and CpG methylation data.

FIG. 2 is a photograph showing construction of an ovarian cancer metastasis animal model by injecting SK-OV-3 cell line into the intraperitoneal cavity of a nude mouse.

FIG. 3 is the result of showing the distribution patterns of global DNA methylation in primary ovarian cancer cell line (SK-OV-3) and tumor tissues of 7 animals with ovarian cancer metastasis (n=7; designated as 1C˜8C).

FIG. 4 is the Heatmap result of the genes showing significant expression changes in the metastatic tumor tissues, compared to the primary ovarian cancer cell line.

FIG. 5 is the result of showing changes in the DNA methylation and gene expression in ovarian cancer metastasis animal model.

FIG. 6 is the result of qRT-PCR showing changes in AGR2 gene expression in the tumor tissues of ovarian cancer metastasis animal models (n=7; designated as 1C˜8C).

FIG. 7 is the result of qRT-PCR showing changes in CA9 gene expression in the tumor tissues of ovarian cancer metastasis animal models (n=7; designated as 1C˜8C).

FIG. 8 is the result of qRT-PCR showing changes in GABRP gene expression in the tumor tissues of ovarian cancer metastasis animal models (n=7; designated as 1C˜8C).

FIG. 9 is the result of qRT-PCR showing changes in IFITM1 gene expression in the tumor tissues of ovarian cancer metastasis animal models (n=7; designated as 1C˜8C).

FIG. 10 is the result of qRT-PCR showing changes in MUC13 gene expression in the tumor tissues of ovarian cancer metastasis animal models (n=7; designated as 1C˜8C).

FIG. 11 is the result of DNA methylation microarray for analyzing DNA methylation at the promoter CpG site of AGR2 gene in the tumor tissues of ovarian cancer metastasis animal models (n=7; designated as 1C˜8C).

FIG. 12 is the result of DNA methylation microarray for analyzing DNA methylation at the promoter CpG site of CA9 gene in the tumor tissues of ovarian cancer metastasis animal models (n=7; designated as 1C˜8C).

FIG. 13 is the result of DNA methylation microarray for analyzing DNA methylation at the promoter CpG site of GABRP gene in the tumor tissues of ovarian cancer metastasis animal models (n=7; designated as 1C˜8C).

FIG. 14 is the result of DNA methylation microarray for analyzing DNA methylation at the promoter CpG site of IFITM1 gene in the tumor tissues of ovarian cancer metastasis animal models (n=7; designated as 1C˜8C).

FIG. 15 is the result of DNA methylation microarray for analyzing DNA methylation at the promoter CpG site of MUC13 gene in the tumor tissues of ovarian cancer metastasis animal models (n=7; designated as 1C˜8C).

FIG. 16 is the result of analyzing changes in AGR2 gene expression after treatment of SK-OV-3 cell line with 5-aza-2′-deoxycytidine.

FIG. 17 is the result of analyzing changes in CA9 gene expression after treatment of SK-OV-3 cell line with 5-aza-2′-deoxycytidine.

FIG. 18 is the result of analyzing changes in GABRP gene expression after treatment of SK-OV-3 cell line with 5-aza-2′-deoxycytidine.

FIG. 19 is the result of analyzing changes in IFITM1 gene expression after treatment of SK-OV-3 cell line with 5-aza-2′-deoxycytidine.

FIG. 20 is the result of analyzing changes in MUC13 gene expression after treatment of SK-OV-3 cell line with 5-aza-2′-deoxycytidine.

### DETAILED DESCRIPTION OF THE EMBODIMENTS

Based on the finding that the specific CpG sites of AGR2, CA9, GABRP, IFITM1 and MUC13 gene promoters are specifically hypomethylated in metastatic ovarian cancer tissues, the present invention provides a technique of diagnosing ovarian cancer metastasis or predicting the risk of metastasis by using the methylation levels of the promoters of these genes as biomarkers.

Because the CpG sites of AGR2, CA9, GABRP, IFITM1 and MUC13 gene promoters are specifically hypomethylated in metastatic ovarian cancer tissues, respectively, each of them can be used as a single biomarker for diagnosing ovarian cancer metastasis or predicting the risk of metastasis, or two or more thereof can be used as multi-biomarkers.

Accordingly, in one aspect, the present invention relates to a composition for diagnosing ovarian cancer metastasis or predicting the risk of metastasis including an agent measuring methylation levels at the CpG sites of one or more gene promoters selected from the group consisting of AGR2, CA9, GABRP, IFITM1 and MUC13, and a kit including the same.

In one preferred embodiment, the present invention relates to a composition for diagnosing ovarian cancer metastasis or predicting the risk of metastasis including an agent measuring the methylation level at the CpG site of AGR2 gene promoter, and a kit including the same.

In this case, more preferably, the composition and the kit may further include an agent measuring methylation levels at the CpG sites of one or more gene promoters selected from the group consisting of CA9, GABRP, IFITM1 and MUC13.

In another preferred embodiment, the present invention relates to a composition for diagnosing ovarian cancer metastasis or predicting the risk of metastasis including an agent measuring the methylation level at the CpG site of CA9 gene promoter, and a kit including the same.

In this case, more preferably, the composition and the kit may further include an agent measuring methylation levels at the CpG sites of one or more gene promoters selected from the group consisting of AGR2, GABRP, IFITM1 and MUC13.

In another preferred embodiment, the present invention relates to a composition for diagnosing ovarian cancer metastasis or predicting the risk of metastasis including an agent measuring the methylation level at the CpG site of GABRP gene promoter, and a kit including the same.

In this case, more preferably, the composition and the kit may further include an agent measuring methylation levels at the CpG sites of one or more gene promoters selected from the group consisting of AGR2, CA9, IFITM1 and MUC13.

In another preferred embodiment, the present invention relates to a composition for diagnosing ovarian cancer metastasis or predicting the risk of metastasis including an agent measuring the methylation level at the CpG site of IFITM1 gene promoter, and a kit including the same.

In this case, more preferably, the composition and the kit may further include an agent measuring methylation levels at the CpG sites of one or more gene promoters selected from the group consisting of AGR2, CA9, GABRP and MUC13.

In another preferred embodiment, the present invention relates to a composition for diagnosing ovarian cancer metastasis or predicting the risk of metastasis including an agent measuring the methylation level at the CpG site of MUC13 gene promoter, and a kit including the same.

In this case, more preferably, the composition and the kit may further include an agent measuring methylation levels at the CpG sites of one or more gene promoters selected from the group consisting of AGR2, CA9, GABRP and IFITM1.

In the present invention, the sequence information of AGR2, CA9, GABRP, IFITM1 and MUC13 genes can be obtained from the known gene database. For example, the nucleic acid sequence of human AGR2 gene can be obtained from GenBank Accession NO. NM_{—}006408, the nucleic acid sequence of human CA9 gene can be obtained from GenBank Accession NO. NM_{—}001216, the nucleic acid sequence of human GABRP gene can be obtained from GenBank Accession NO. NM_{—}014211, the nucleic acid sequence of human IFITM1 gene can be obtained from GenBank Accession NO. NM_{—}003641, and the nucleic acid sequence of human MUC13 gene can be obtained from GenBank Accession NO. NM_{—}033049.

As used herein, the term “methylation” refers to attachment of methyl groups to bases constituting genomic DNA. Preferably, the methylation, as used herein, means methylation that occurs at cytosines of specific CpG sites in a particular gene promoter. If methylation occurs, binding of transcription factors is inhibited to suppress expression of a particular gene. If non-methylation or hypomethylation occurs, expression of the particular gene is increased.

In the genomic DNA of mammalian cells, there is the fifth base in addition to A, C, G and T, namely, 5-methylcytosine (5-mC), in which a methyl group is attached to the fifth carbon of the cytosine ring. Methylation of 5-methylcytosine is always attached only to the C of a CG dinucleotide (5′-mCG-3′), which is frequently marked CpG. The methylation of this CpG inhibits a repetitive sequence in genomes, such as Alu or transposon, from being expressed. Also, 5-mC of this CpG is naturally deaminated to thymine (T), and thus CpG is a site where an epigenetic change in mammalian cells appears most often.

As used herein, the phrase “measuring the methylation level” means to determine the methylation level of gene promoter, and the methylation level can be determined by methylation-specific PCR, for example, methylation-specific PCR (methylation-specific polymerase chain reaction, MSP), real time methylation-specific PCR (real time methylation-specific polymerase chain reaction), PCR using a methylation DNA-specific binding protein, and quantitative PCR. Alternatively, it can be determined by automatic sequencing such as pyrosequencing and bisulfite sequencing, but is not limited thereto.

Preferably, measurement of the methylation level at the CpG site of the AGR2 gene promoter in the present invention may mean measurement of the methylation level of cytosine at the CpG site from the base 16844546 to 16844667 of chromosome 7. In the present invention, the base 16844546 to 16844667 of chromosome 7 is represented by SEQ ID NO. 1.

More preferably, measurement of the methylation level at the CpG site of the AGR2 gene promoter in the present invention may mean measurement of the methylation level of cytosine at the position 16844606 (at position 61 of SEQ ID NO. 1) of chromosome 7.

Preferably, measurement of the methylation level at the CpG site of the CA9 gene promoter in the present invention may mean measurement of the methylation level of cytosine at the CpG site from the base 35673849 to 35673970 of chromosome 9. In the present invention, the base 35673849 to 35673970 of chromosome 9 is represented by SEQ ID NO. 2.

More preferably, measurement of the methylation level at the CpG site of the CA9 gene promoter in the present invention may mean measurement of the methylation level of cytosine at the position 35673909 (at position 61 of SEQ ID NO. 2) of chromosome 9.

Preferably, measurement of the methylation level at the CpG site of the GABRP gene promoter in the present invention may mean measurement of the methylation level of cytosine at the CpG site from the base 170209700 to 170209821 or from the base 170209521 to 170209642 of chromosome 5. In the present invention, the base sequences of the CpG site are represented by SEQ ID NO. 3 and SEQ ID NO. 4, respectively.

More preferably, measurement of the methylation level at the CpG site of the GABRP gene promoter in the present invention may mean measurement of the methylation level of cytosine at the position 170209760 (at position 61 of SEQ ID NO. 3) or at the position 170209581 (at position 61 of SEQ ID NO. 4) of chromosome 5.

Preferably, measurement of the methylation level at the CpG site of the IFITM1 gene promoter in the present invention may mean measurement of the methylation level of cytosine at the CpG site from the base 313984 to 314105 of chromosome 11. In the present invention, the base sequence of the CpG site is represented by SEQ ID NO. 5.

More preferably, measurement of the methylation level at the CpG site of the IFITM1 gene promoter in the present invention may mean measurement of the methylation level of cytosine at the position 314044 (at position 61 of SEQ ID NO. 5) of chromosome 11.

Preferably, measurement of the methylation level at the CpG site of the MUC13 gene promoter in the present invention may mean measurement of the methylation level of cytosine at the CpG site from the base 124653658 to 124653779 or from the base 124653599 to 124653720 of chromosome 3. In the present invention, the base sequences of the CpG site are represented by SEQ ID NOs. 6 and 7, respectively.

More preferably, measurement of the methylation level at the CpG site of the MUC13 gene promoter in the present invention may mean measurement of the methylation level of cytosine at the position 124653718 (at position 61 of SEQ ID NO. 6) or at the position 124653659 (at position 61 of SEQ ID NO. 7) of chromosome 3.

In the present invention, the base sequences of the human genomic chromosomes are given according to the latest February 2009 Human reference sequence (GRCh37), but the specific sequences of the human genomic chromosomes can be slightly revised according to update of the genomic sequence analysis. The annotation of the human genomic locations of the present invention may differ depending on the revision. Therefore, although the annotation of the human genomic locations according to the February 2009 Human reference sequence (GRCh37) is revised according to the human reference sequence updated after the filing date of the present application, it will be apparent that the revised annotation of human genomic locations is also within the scope of the present invention. Such revision may be readily apparent to those skilled in the art to which the present invention pertains.

Based on the finding that there are differences in gene expressions between primary tumors at the early stage and metastatic tumors, the present inventors compared the gene expression patterns between primary cancer cell lines and metastatic tissues to finally select genes, in which CpG methylation in their promoter regions was found to affect gene expressions, from the genes showing gene expression changes in metastatic tissues. Furthermore, they identified the specific CpG sites that affect the gene expressions, and also found that ovarian cancer metastasis and the risk of metastasis can be predicted by measuring methylation levels at the specific CpG sites of the corresponding gene promoters.

In more detail, the present inventors constructed ovarian cancer metastasis animal model by injecting the primary ovarian cancer cell line SK-OV-3 into the intraperitoneal cavity of 10 nude mice, and they extracted genomic DNAs and RNAs from the tumor tissues of these animal models to carry out DNA methylation microarray using an Illumina Human Methylation 450 Bead Chip and gene expression microarray using an Affymetrix Human Gene 1.0 ST. Through the integration analysis of the results, they selected genes, in which changes in CpG methylation in their promoter regions were suspected to affect gene expressions.

Of the selected genes, the ovarian cancer metastasis mouse model showed up to 26-188-fold increase in AGR2 expression, and about 3.5-7-fold decrease in DNA methylation, compared to the primary cancer cell line. The ovarian cancer metastasis mouse model showed up to 54.4-372.4-fold increase in CA9 expression, and about 1.6-7.0-fold decrease in DNA methylation at the specific CpG site of the promoter, compared to the primary cancer cell line. The ovarian cancer metastasis mouse model showed up to 17.3-86.6-fold increase in GABRP expression, and about 4.7-6.6-fold decrease in DNA methylation at the specific CpG site of the promoter, compared to the primary cancer cell line. The ovarian cancer metastasis mouse model showed up to 4.5-9.5-fold increase in IFITM1 expression, and about 2.5-3.8-fold decrease in DNA methylation at the specific CpG site of the promoter, compared to the primary cancer cell line. The ovarian cancer metastasis mouse model showed up to 3.4-68.8-fold increase in MUC13 expression, and about 1.8-2.0-fold decrease in DNA methylation at the specific CpG site of the promoter, compared to the primary cancer cell line.

Further, treatment of the primary cell line SKOV-3 with a DNA demethylating agent, 5-aza-2′-deoxycytidine resulted in about 2-fold increase in AGR2 gene expression, about 3.4-fold increase in CA9 gene expression, about 1.8-fold increase in GABRP gene expression, about 1.7-fold increase in IFITM1 gene expression, and 17.8-fold increase in MUC13 gene expression, indicating that expressions of the above genes are regulated by DNA methylation.

Therefore, hypomethylation of DNA methylation at the specific CpG site of AGR2, GABRP, IFITM1 and/or MUC13 can be utilized as biomarkers for diagnosing ovarian cancer metastasis or predicting the risk of metastasis.

As used herein, the term “diagnosis of metastasis” means examination of ovarian cancer metastasized to other tissues from the ovary. In general, ovarian cancer spreads to other organ tissues through the peritoneal cavity. The tissues other than the ovary may be, for example, various organ tissues within the peritoneal cavity including the large intestine, small intestine, and periphery of the liver. More preferably, diagnosis of metastasis, as used herein, means examination of metastatic status of ovarian cancer by distinguishing a sample of a patient with metastasis from the non-metastatic, primary ovarian cancer sample.

As used herein, the term “prediction of the risk of metastasis” or “diagnosis of the risk of metastasis” means prediction of possibility of ovarian cancer spreading from the ovary to other tissues. More preferably, the prediction of the risk of metastasis, as used herein, means prediction of possibility of recurrence and metastasis of ovarian cancer in the treated tissue after a patient with metastatic ovarian cancer is treated with therapy such as surgery, radiation therapy, chemotherapy or the like. From another point of view, the prediction of the risk of metastasis, as used herein, means prediction of possibility of metastasis in a patient with ovarian cancer by distinguishing a sample of the patient at the risk of the metastasis from the non-metastatic, primary ovarian cancer sample.

Further, aberrant methylation in cancer tissues is considerably similar to methylation of genomic DNA obtained from a biological sample such as cells, whole blood, serum, plasma, saliva, sputum or urine. Therefore, when the markers of the present invention are used, there is an advantage that it is possible to diagnose ovarian cancer metastasis or predict the risk of metastasis in the blood or body fluid in a simple manner.

In the present invention, the agent measuring a methylation level at the CpG site may include a compound modifying an unmethylated cytosine base or a methylation-sensitive restriction enzyme, primers specific to the methylated allele sequence of AGR2, GABRP, IFITM1 and/or MUC13 gene, and primers specific to the unmethylated allele sequence of the gene.

The compound modifying an unmethylated cytosine base may be bisulfite, but is not limited thereto, preferably sodium bisulfite. A method of detecting promoter methylation by modifying the unmethylated cytosine residue using bisulfite is widely known in the art (WO01/26536; US2003/0148326A1).

Further, the methylation-sensitive restriction enzyme is a restriction enzyme capable of specifically detecting CpG methylation, and preferably a restriction enzyme including CG as a restriction enzyme recognition site. Examples thereof include SmaI, SacII, EagI, HpaII, MspI, BssHII, BstUI, NotI or the like, but are not limited thereto. Cleavage by a restriction enzyme differs depending on methylation or unmethylation of C at the restriction enzyme recognition site, and the methylation can be detected by PCR or Southern blot analysis. In addition to the restriction enzymes, other methylation-sensitive restriction enzymes are well known in the art.

The methylation level of the particular CpG site of AGR2, GABRP, IFITM1 and/or MUC13 gene promoter in an individual suspected of having ovarian cancer metastasis may be determined by obtaining genomic DNA from a biological sample of the individual, treating the obtained DNA with a compound modifying an unmethylated cytosine base or a methylation-sensitive restriction enzyme, amplifying the treated DNA using primers by PCR, and then identifying the presence of the resulting amplified product.

Therefore, the agent of the present invention may include primers specific to the methylated allele sequence of AGR2, GABRP, IFITM1 and/or MUC13 gene, and primers specific to the unmethylated allele sequence of the gene. As used herein, the term “primer” means a short nucleic acid sequence having a free 3′ hydroxyl group, which is able to form base-pairing interaction with a complementary template and serves as a starting point for replication of the template strand. A primer is able to initiate DNA synthesis in the presence of a reagent for polymerization (i.e., DNA polymerase or reverse transcriptase) and four different nucleoside triphosphates at suitable buffers and temperature. In addition, the primers are sense and antisense nucleic acids having a sequence of 7 to 50 nucleotides. The primer may have additional properties that do not change the nature of the primer to serve as a starting point for DNA synthesis.

The primers of the present invention can be designed according to the CpG sequence that is subjected to methylation analysis, and may be a set of primers that are able to specifically amplify bisulfite-unmodified cytosine due to methylation and a set of primers that are able to specifically amplify bisulfite-modified cytosine due to unmethylation.

The diagnostic composition for ovarian cancer metastasis may further include polymerase, agarose, and a buffer solution for electrophoresis, in addition to the above agent.

In another aspect, the present invention relates to a method for diagnosing ovarian cancer metastasis or the risk of metastasis by measuring methylation levels at the specific CpG sites of one or more gene promoters selected from the group consisting of AGR2, CA9, GABRP, IFITM1 and MUC13.

For example, the present invention relates to a method for diagnosing ovarian cancer metastasis or the risk of metastasis, including the steps of:

(a) measuring methylation levels at the CpG sites of one or more gene promoters selected from the group consisting of AGR2, CA9, GABRP, IFITM1 and MUC13 in a biological sample of a subject,

(b) comparing the methylation levels with those of the gene promoters of a control sample, and

(c) determining that the subject has ovarian cancer metastasis or is at the risk of the metastasis when the methylation levels measured in the sample of the subject are lower than those of the control sample.

Preferably, the control sample may be a sample of a subject with non-metastatic ovarian cancer, or a control sample of primary ovarian cancer.

In one preferred embodiment, the present invention relates to a method for diagnosing ovarian cancer metastasis or the risk of metastasis, including the steps of:

measuring methylation level at the CpG site of AGR2 gene promoter in a biological sample of a subject,

comparing the methylation level with that of the gene promoters of a control sample, and

determining that the subject has ovarian cancer metastasis or is at the risk of the metastasis when the methylation level measured in the sample of the subject is lower than that of the control sample.

In this case, more preferably, the method may further include the steps of:

measuring methylation levels at the CpG sites of one or more gene promoters selected from the group consisting of CA9, GABRP, IFITM1 and MUC13 in a biological sample of a subject,

comparing the methylation levels with those of the gene promoters of a control sample, and

determining that the subject has ovarian cancer metastasis or is at the risk of the metastasis when the methylation levels measured in the sample of the subject are lower than those of the control sample.

In another preferred embodiment, the present invention relates to a method for diagnosing ovarian cancer metastasis or the risk of metastasis, including the steps of:

measuring methylation level at the CpG site of CA9 gene promoter in a biological sample of a subject,

comparing the methylation level with that of the gene promoters of a control sample, and

determining that the subject has ovarian cancer metastasis or is at the risk of the metastasis when the methylation level measured in the sample of the subject is lower than that of the control sample.

In this case, more preferably, the method may further include the steps of:

measuring methylation levels at the CpG sites of one or more gene promoters selected from the group consisting of AGR2, GABRP, IFITM1 and MUC13 in a biological sample of a subject,

comparing the methylation levels with those of the gene promoters of a control sample, and

determining that the subject has ovarian cancer metastasis or is at the risk of the metastasis when the methylation levels measured in the sample of the subject are lower than those of the control sample.

In another preferred embodiment, the present invention relates to a method for diagnosing ovarian cancer metastasis or the risk of metastasis, including the steps of:

measuring methylation level at the CpG site of GABRP gene promoter in a biological sample of a subject,

comparing the methylation level with that of the gene promoters of a control sample, and

determining that the subject has ovarian cancer metastasis or is at the risk of the metastasis when the methylation level measured in the sample of the subject is lower than that of the control sample.

In this case, more preferably, the method may further include the steps of:

measuring methylation levels at the CpG sites of one or more gene promoters selected from the group consisting of AGR2, CA9, IFITM1 and MUC13 in a biological sample of a subject,

comparing the methylation levels with those of the gene promoters of a control sample, and

determining that the subject has ovarian cancer metastasis or is at the risk of the metastasis when the methylation levels measured in the sample of the subject are lower than those of the control sample.

In another preferred embodiment, the present invention relates to a method for diagnosing ovarian cancer metastasis or the risk of metastasis, including the steps of:

measuring methylation level at the CpG site of IFITM1 gene promoter in a biological sample of a subject,

comparing the methylation level with that of the gene promoters of a control sample, and

determining that the subject has ovarian cancer metastasis or is at the risk of the metastasis when the methylation level measured in the sample of the subject is lower than that of the control sample.

In this case, more preferably, the method may further include the steps of:

measuring methylation levels at the CpG sites of one or more gene promoters selected from the group consisting of AGR2, CA9, GABRP and MUC13 in a biological sample of a subject,

comparing the methylation levels with those of the gene promoters of a control sample, and

determining that the subject has ovarian cancer metastasis or is at the risk of the metastasis when the methylation levels measured in the sample of the subject are lower than those of the control sample.

In another preferred embodiment, the present invention relates to a method for diagnosing ovarian cancer metastasis or the risk of metastasis, including the steps of:

measuring methylation level at the CpG site of MUC13 gene promoter in a biological sample of a subject,

comparing the methylation level with that of the gene promoters of a control sample, and

determining that the subject has ovarian cancer metastasis or is at the risk of the metastasis when the methylation level measured in the sample of the subject is lower than that of the control sample.

In this case, more preferably, the method may further include the steps of:

measuring methylation levels at the CpG sites of one or more gene promoters selected from the group consisting of AGR2, CA9, GABRP and IFITM1 in a biological sample of a subject,

comparing the methylation levels with those of the gene promoters of a control sample, and

determining that the subject has ovarian cancer metastasis or is at the risk of the metastasis when the methylation levels measured in the sample of the subject are lower than those of the control sample.

As used herein, the term “biological sample” includes samples displaying a difference in the methylation levels of AGR2, GABRP, IFITM1 and/or MUC13 gene by the ovarian cancer metastasis, such as tissues, cells, whole blood, serum, plasma, saliva, sputum, cerebrospinal fluid or urine, but is not limited thereto.

First, to measure the methylation level of genomic DNAs obtained from the individuals suspected of having ovarian cancer metastasis, the genomic DNAs can be obtained by a phenol/chloroform extraction method, an SDS extraction method, a CTAB separation method typically used in the art, or using a commercially available DNA extraction kit.

In the present invention, the step of (a) measuring methylation levels at the CpG sites of gene promoters may be performed by using a compound modifying an unmethylated cytosine base or a methylation sensitive restriction enzyme, primers specific to the methylated sequence of the gene promoter, and primers specific to the unmethylated sequence.

In more detail, the step may be performed by a step of treating the genomic DNA obtained from the sample with the compound modifying an unmethylated cytosine base or the methylation sensitive restriction enzyme; and a step of measuring the methylation level of the treated DNA by one or more methods selected from the group consisting of methylation-specific polymerase chain reaction, real time methylation-specific polymerase chain reaction, PCR using a methylated DNA-specific binding protein, quantitative PCR, pyrosequencing and bisulfite sequencing using primers capable of amplifying the methylated region of the gene promoter.

The compound modifying unmethylated cytosine base may be bisulfite, and preferably sodium bisulfite. The method of detecting promoter methylation by modifying unmethylated cytosine residues using bisulfite is widely known in the art.

The methylation-sensitive restriction enzyme is, as described above, a restriction enzyme capable of specifically detecting the methylation of the particular CpG site, and preferably a restriction enzyme containing CG as a restriction enzyme recognition site. Examples thereof include SmaI, SacII, EagI, HpaII, MspI, BssHII, BstUI, NotI or the like, but are not limited thereto.

The primers used herein are, as described above, designed according to the particular CpG site that is subjected to methylation analysis, and may be a set of primers that are able to specifically amplify bisulfite-unmodified cytosine due to methylation and a set of primers that are able to specifically amplify bisulfite-modified cytosine due to unmethylation.

The step of measuring the methylation level of the particular CpG site may be conducted by a method known in the art. For example, electrophoresis is performed to detect the presence of a band at the desired size. For example, in the case of using the compound modifying the unmethylated cytosine residues, methylation can be determined according to the presence of the PCR product that is amplified by the two types of primer pairs, that is, the set of primers that are able to specifically amplify bisulfite-unmodified cytosine due to methylation and a set of primers that are able to specifically amplify bisulfite-modified cytosine due to unmethylation. Preferably, methylation can be determined by treating genomic DNA of a sample with bisulfite, amplifying the CpG site of AGR2, GABRP, IFITM1 and/or MUC13 gene by PCR, and then analyzing the amplified base sequence by bisulfite genomic sequencing.

Further, if a restriction enzyme is used, methylation can be determined by a method known in the art. For example, when the PCR product is present in the restriction enzyme-treated DNA, under the state where the PCR product is present in the mock DNA, it is determined as promoter methylation. When the PCR product is absent in the restriction enzyme-treated DNA, it is determined as promoter unmethylation. Accordingly, the methylation can be determined, which is apparent to those skilled in the art. The term ‘mock DNA’ refers to a sample DNA isolated from clinical samples with no treatment.

When hypomethylations at the CpG sites of AGR2, CA9, GABRP, IFITM1 and/or MUC13 gene promoters are observed in the sample of the subject by the above method, it can be predicted that the subject has ovarian cancer metastasis or is at the risk of the metastasis.

Therefore, the method of providing information for the diagnosis of ovarian cancer metastasis of the present invention is used to effectively examine the methylation of AGR2, GABRP, IFITM1 and/or MUC13 gene promoter, thereby diagnosing ovarian cancer metastasis or predicting the risk of metastasis.

### DETAILED DESCRIPTION OF THE EMBODIMENTS

Hereinafter, the present invention will be described in more detail with reference to Examples. However, the following Examples are for illustrative purposes only, and the present invention is not intended to be limited thereto.

### Example 1

### Cell Line and Ovarian Cancer Metastasis Mouse Model

Human ovarian cancer cell line SK-OV-3 was purchased from American type culture collection (ATCC no. HTB-77) and cultured in a McCoy's 5a medium containing 10% FBS (fetal bovine serum), 100 U/mL penicillin and 100 μg/mL streptomycin.

In order to prepare ovarian cancer metastasis mouse model, 2×10⁶ SK-OV-3 cells were suspended in the cell culture medium, and injected into the peritoneal cavity of 10 4-6-week old female BALB/c nude mice. 4 weeks later, tumor tissues (organ tissues in the peritoneal cavity, including the large intestine, small intestine, and periphery of the liver) formed by migration of the cell line along the peritoneal cavity were excised and stored in liquid nitrogen.

### Example 2

### Total RNA Extraction

Total RNAs were extracted from SK-OV-3 cell line and the tumor tissues using an RNeasy mini kit (Qiagen), respectively. The extraction was performed according to manufacturer's instructions. The extracted total RNAs were quantified using a spectrophotometer, and RNA degradation was examined by electrophoresis in a 1% agarose gel.

### Example 3

### Quantitative Real-Time PCR (qRT-PCR)

For cDNA synthesis, Superscript II reverse transcriptase (Invitrogen) was used. 1 μg of total RNA and 50 ng of oligo dT were denatured at 70° C. for 10 minutes, and then mixed with a reaction mixture containing 4 μl of 5×RT buffer, 2 μl of 0.1 mM DTT, 4 μl of 2.5 mM dNTP mixture, 200 units of Superscript II reverse transcriptase and 10 units of RNase inhibitor to prepare 20 μl of the resulting reaction mixture, which was reacted at 25° C. for 10 minutes, at 42° C. for 50 minutes, and at 95° C. for 5 minutes to synthesize cDNA. This cDNA was diluted at 1:4, and 2 μl thereof was used as a template for qRT-PCR. In qRT-PCR, 20 μl of reaction mixture containing 2 μl of cDNA, 10 μl of SYBR Premix EX Taq (Takara Bio), 0.4 μl of Rox reference dye (50×, Takara Bio), and 200 nM of primers of each gene was reacted at 95° C. for 30 seconds, and then repeated for 40 cycles (at 95° C. for 3 seconds, and at 60° C. for 30 seconds) using an ABI 7500fast sequence detection system (Applied Biosystems) for amplification. The PCR products were reacted at 95° C. for 15 seconds, at 60° C. for 1 minute, and at 95° C. for 15 seconds to examine their specificity. 18S rRNA expression was used as an internal control, and expressions of AGR2, CA9, GABRP, IFITM1 and MUC13 genes were normalization using the 18S rRNA expression level by a ΔΔC_{T} method. The sequences of the primers used are as follows.

TABLE 1

| | | SEQ |
|---|---|---|
| | Sequence | ID NO. |
| human AGR2 (forward) | 5′-AGTTTGTCCTCCTCAATCTGGTTT-3′ | 8 |
| human AGR2 (reverse) | 5′-GACATACTGGCCATCAGGAGAAA-3′ | 9 |
| human CA9 (forward) | 5′-TGACTCTCGGCTACAGCTGAACT-3′ | 10 |
| human CA9 (reverse) | 5′-CCACTCCAGCAGGGAAGGA-3′ | 11 |
| human GABRP (forward) | 5′-CTCGATTCAGTCCCTGCAAGA-3′ | 12 |
| human GABRP (reverse) | 5′-GTGCGGGACCCGATCAT-3′ | 13 |
| human IFITM1 (forward) | 5′-CGCCAAGTGCCTGAACATCT-3′ | 14 |
| human IFITM1 (reverse) | 5′-TACCAGTAACAGGATGAATCCAATG-3′ | 15 |
| human MUC13 (forward) | 5′-AGAAACATTCCATGGCCTATCAA-3′ | 16 |
| human MUC13 (reverse) | 5′-TGTCCATAAACAGATGTGCCAAA-3′ | 17 |
| human 18S rRNA (forward) | 5′-CGGCTACCACATCCAAGGAA-3′ | 18 |
| human 18S rRNA (reverse) | 5′-GCTGGAATTACCGCGGCT-3′ | 19 |

### Example 4

### 5-Aza-2′-deoxycytidine (5-aza-dC) treatment

SK-OV-3 cell line was treated with a methylation inhibitor, 5-aza-2′-deoxycytidine (Sigma-Aldrich) at concentrations of 5 and 10 μM for 3 days, and then changes in AGR2, CA9, GABRP, IFITM1, and MUC13 gene expressions were measured by qRT-PCP.

### Example 5

### mRNA Microarray

mRNA microarray was performed using a GeneChip Human Gene 1.0 ST arrays.

Gene expression values obtained after scanning were subjected to background correction, RMA normalization (Biostatistics. 2003 April; 4(2):249-64. Exploration, normalization, and summaries of high density oligonucleotide array probe level data), and log 2 transformation, and finally used for statistical analysis. In order to identify differentially expressed genes (DEGs) in two groups, Bayesian t-test (Limma: Linear Models for. Microarray Data. Gordon K. Smyth.) was used. Finally, genes with p value<0.05 and absolute value of log 2 (fold change) greater than 0.585 were selected as DEG.

### Example 6

### DNA Methylation Microarray

DNA methylation microarray was performed using an Infinium® Human Methylation 450K BeadChip. The level of DNA methylation was reported as a β-value ranging from 0 to 1, with 0 being completely unmethylated and 1 being completely methylated at the corresponding CpG site.

In order to identify differentially methylated genes (DMGs) in two groups, Bayesian t-test was used. Finally, the CpG sites with p value<0.05 and absolute β-value difference=0.3 were selected as differentially methylated CpG sites, and of them, genes showing methylation changes at the CpG sites in the promoter regions were selected as DMG.

### Example 7

### Integration of DEG and DMG Data

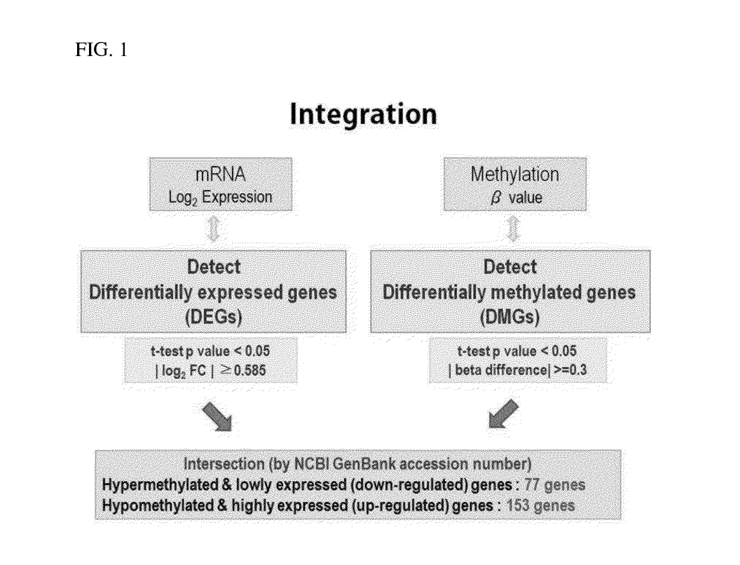

According to the procedure of FIG. 1, DEG and DMG data thus determined were integrated.

### Experimental Results

1. Construction of Ovarian Cancer Metastasis Animal Model

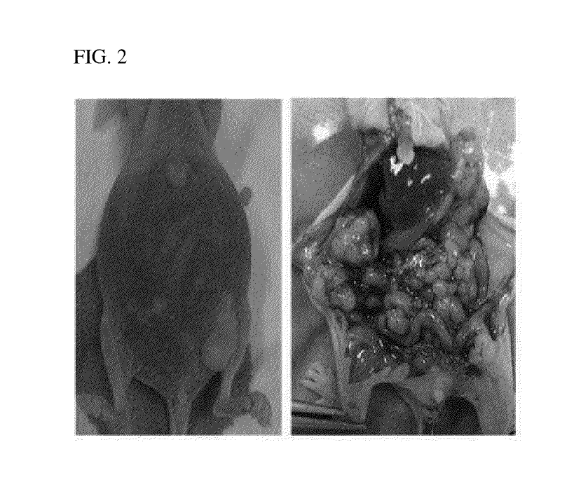

Ovarian cancer metastasis animal models were constructed by injecting the ovarian cancer cell line SK-OV-3 into the intraperitoneal cavity of 10 female nude mice (FIG. 2).

2. Analysis of Epigenetic Change in Ovarian Cancer Metastasis Animal Model

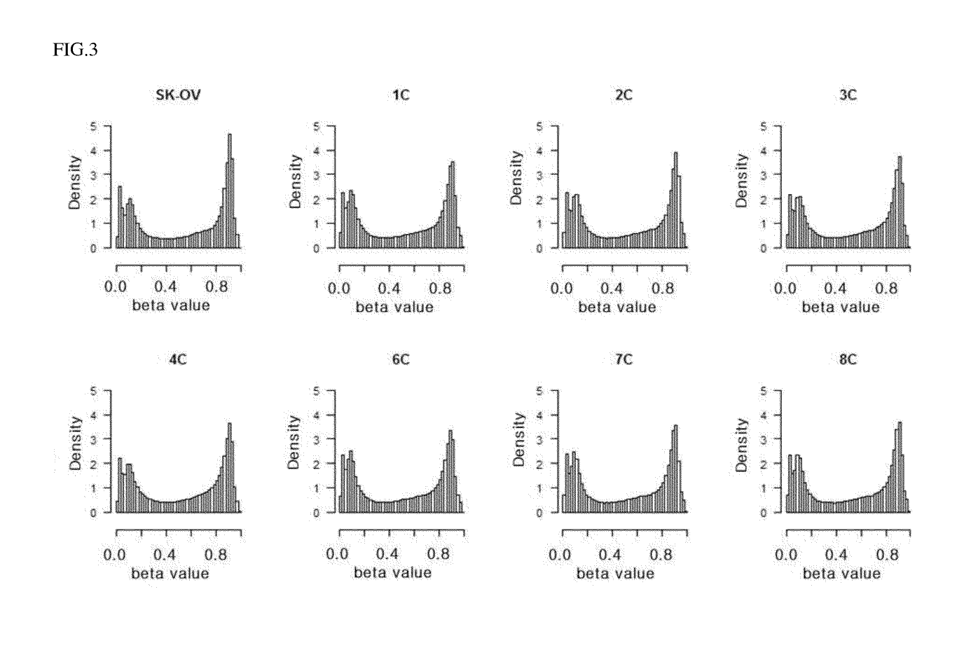

Genomic DNAs were extracted from the tumor tissues (organ tissues in the peritoneal cavity, including the large intestine, small intestine, and periphery of the liver) obtained from metastasis animal model and the ovarian cancer cell line SK-OV-3, and subjected to DNA methylation microarray using an Illumina Human Methylation 450 BeadChip, thereby analyzing CpG sites showing significant changes in DNA methylation in metastatic tumor tissues, compared to the primary ovarian cancer cell line. As a result, decreased global DNA methylation (global hypomethylation) was observed in the metastatic tumor tissues, compared to the primary ovarian cancer cell line (FIG. 3).

3. Analysis of Gene Expression Changes in Ovarian Cancer Metastasis Animal Model

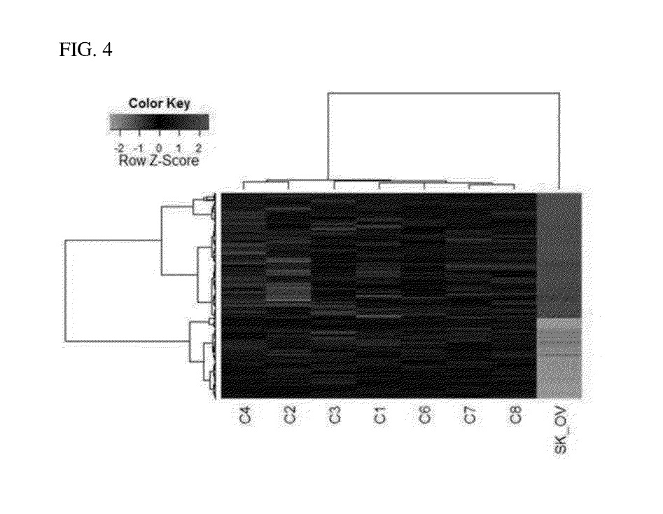

RNAs were extracted from the tumor tissues obtained from metastasis animal model and the ovarian cancer cell line SK-OV-3, and subjected to expression microarray using an Affymetrix Human Gene 1.0 ST, thereby analyzing genes showing significant changes in their expression in metastatic tumor tissues, compared to the primary ovarian cancer cell line (FIG. 4). As a result, expressions of the genes related to cell adhesion, cell cycle, wound healing, and coagulation were increased, whereas expressions of the genes related to transcription, transcriptional regulation, cell death and cell death regulation were remarkably decreased (Table 2).

TABLE 2

| | Cluster | Enrichment | Gene function | | | BH |
|---|---|---|---|---|---|---|
| | No. | Score | (GOTERM_BP_FAT) | Number | P value | p value |
| Increased | Cluster 1 | 8.2 | Cell adhesion | 85 | 2.35E−10 | 1.10E−07 |
| expression | | | Biological adhesion | 85 | 2.52E−10 | 1.01E−07 |
| | Cluster 2 | 7.6 | M phase | 51 | 5.34E−10 | 1.88E−07 |
| | | | Cell cycle | 58 | 1.53E−09 | 4.77E−07 |
| | Cluster 3 | 6.6 | Nucleosome assembly | 27 | 9.48E−13 | 2.67E−09 |
| | | | Chromatin assembly | 27 | 2.36E−12 | 3.31E−09 |
| | Cluster 4 | 5.4 | Calcium-dependent | 11 | 2.70E−07 | 4.22E−05 |
| | | | cell-cell adhesion | | | |
| | | | Extracellular structure | 28 | 9.53E−07 | 1.34E−04 |
| | Cluster 5 | 2.7 | Wound healing | 25 | 3.62E−04 | 0.029 |
| | | | Coagulation | 16 | 8.98E−04 | 0.063 |
| Decreased | Cluster 1 | 5.6 | Transcription | 263 | 2.78E−08 | 1.04E−04 |
| expression | | | Transcriptional | 311 | 1.05E−07 | 1.97E−04 |
| | | | regulation | | | |
| | Cluster 2 | 3.2 | Mitochondria organelle | 29 | 4.72E−05 | 0.029 |
| | | | Protein localization in | 28 | 3.22E−04 | 0.11 |
| | | | cell organelles | | | |
| | Cluster 3 | 3.1 | tRNA metabolism | 27 | 1.96E−05 | 0.018 |
| | | | tRNA aminoacylation | 12 | 0.0025 | 0.27 |
| | Cluster 4 | 3.1 | tRNA metabolism | 27 | 1.96E−05 | 0.018 |
| | | | ncRNA metabolism | 42 | 2.58E−05 | 0.019 |
| | Cluster 5 | 2.5 | Apoptosis regulation | 104 | 3.81E−04 | 0.12 |
| | | | Cell death regulation | 104 | 4.51E−04 | 0.13 |

4. Integrated Analysis of Epigenetic Change and Gene Expression of Ovarian Cancer Metastasis Animal Model

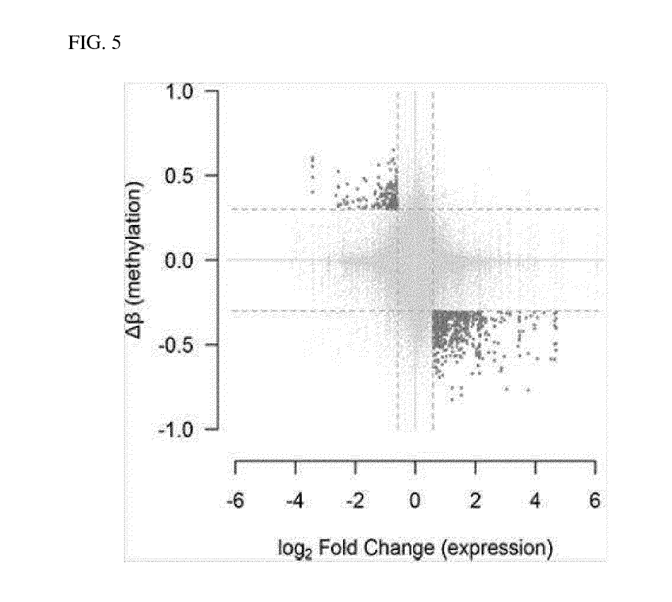

Genes showing changes in DNA methylation and gene expression in metastatic tumor tissues, compared to the primary ovarian cancer cell line, were selected. Integration analysis of the results was performed to select genes, in which changes in methylation at the CpG sites of their promoter regions were suspected to affect gene expression (FIG. 5).

From the results of integration of mRNA expression and CpG methylation data, 153 genes of which expressions were increased by hypomethylation at the CpG sites of the promoters in the metastatic group were selected, and 77 genes of which expressions were decreased by hypermethylation at the CpG sites of the promoters in the metastatic group were selected.

5. Selection of Diagnostic Markers for Ovarian Cancer Metastasis Using Changes in Promoter CpG Methylation

The genes, in which changes in methylation at the CpG sites of their promoter regions were suspected to affect gene expressions, were select by integration analysis, and then genes reported to have functions related to cancer metastasis were secondly selected from the genes. Changes in the gene expressions were examined by Quantitative real-time PCR, so as to select metastasis-specific molecular target candidate genes showing significant differences. Further, the primary cell line SK-OV-3 was treated with a demethylating agent, 5-aza-2′-deoxycytidine, and 5 genes (AGR2, CA9, GABRP, IFITM1, and MUC13) of which expressions were found to be regulated by DNA methylation were finally selected as diagnostic markers for ovarian cancer metastasis using changes in the promoter CpG methylation.

TABLE 3

| | | Expression | | | |
|---|---|---|---|---|---|
| | | logFC | | B | |
| Gene | GenBank | (fold | Expression P | differ- | |
| name | No. | change) | value | ence | β P value |
| AGR2 | NM_006408 | 4.52 | 2.33E−05 | −0.58 | 5.57E−07 |
| CA9 | NM_001216 | 2.96 | 6.64E−05 | −0.57 | 9.06E−09 |
| GABRP | NM_014211 | 2.84 | 2.45E−05 | −0.66 | 4.30E−10 |
| IFITM1 | NM_003641 | 2.36 | 4.37E−07 | −0.36 | 2.56E−06 |
| MUC13 | NM_033049 | 2.65 | 0.00373788 | −0.39 | 2.66E−06 |

6. Changes in Promoter CpG Methylation of the Selected Genes and Changes in Gene Expression in Tumor Tissues of Ovarian Cancer Metastasis Animal Model

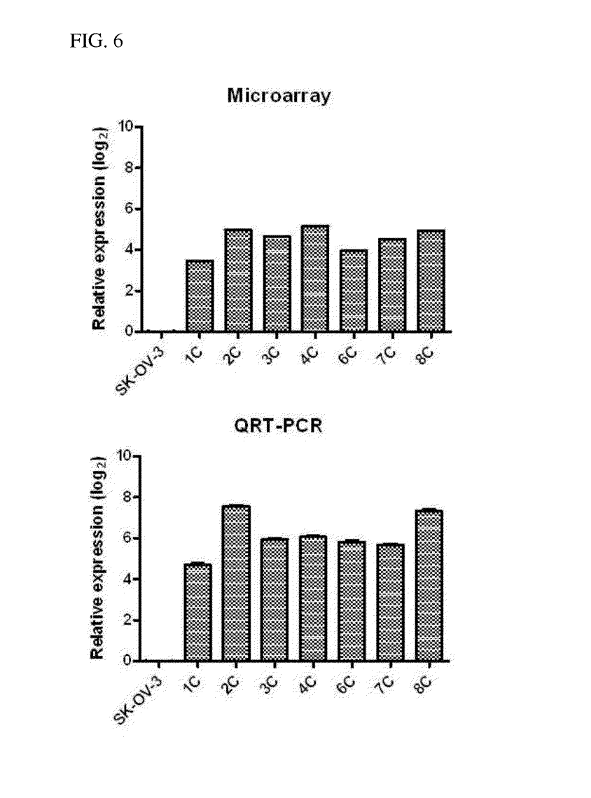

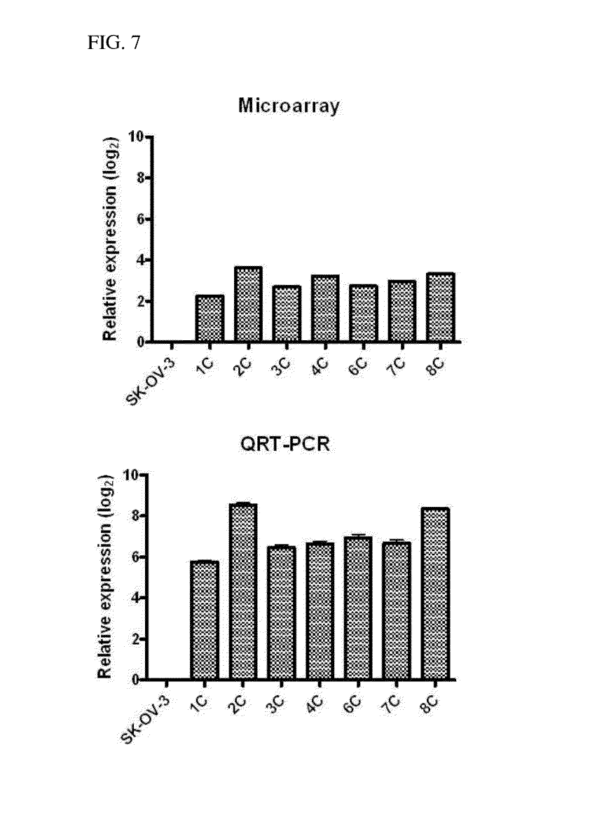

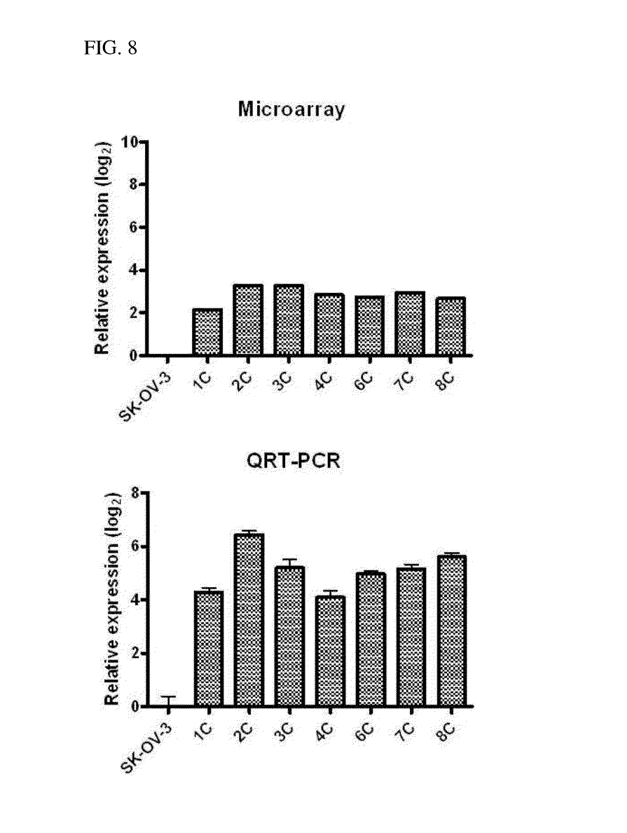

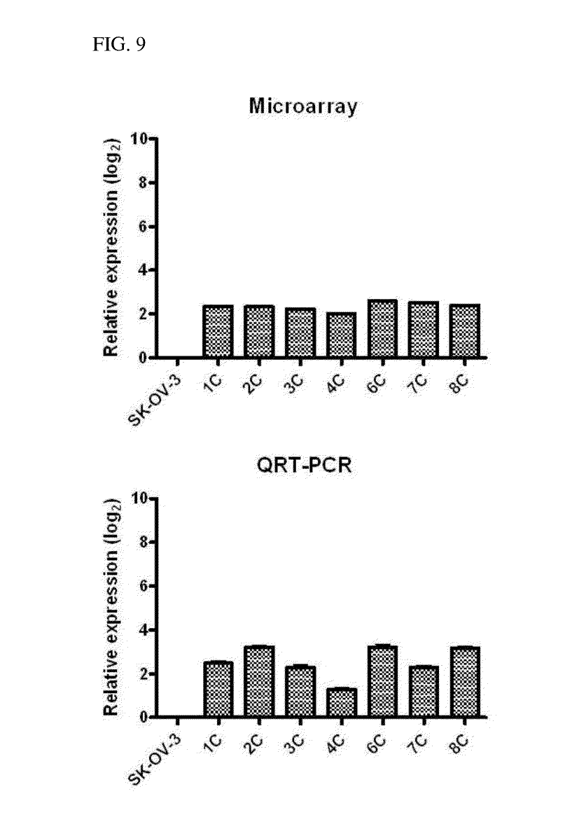

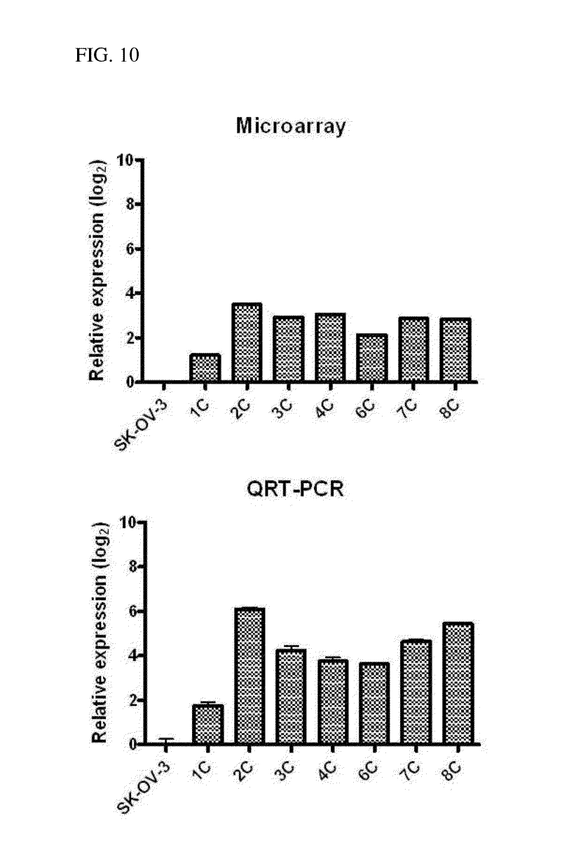

The result of expression microarray showed that expressions of all the five genes (AGR2, CA9, GABRP, IFITM1, and MUC13) were increased in the tumor tissues of ovarian cancer metastasis animal model, and the result of qRT-PCR showed similar expression patterns (FIGS. 6 to 10).

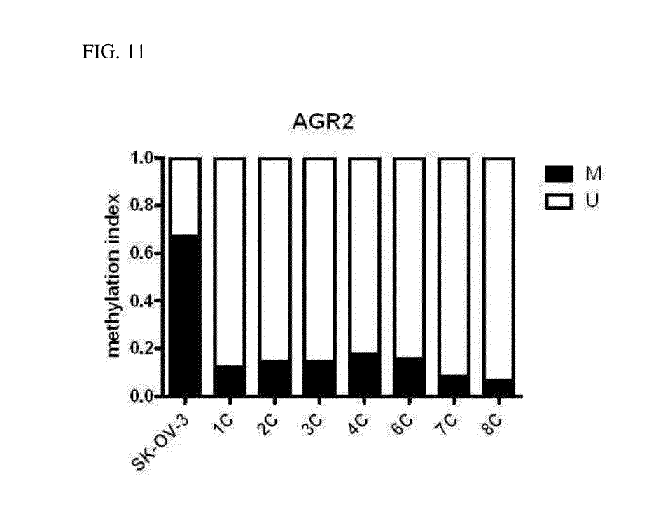

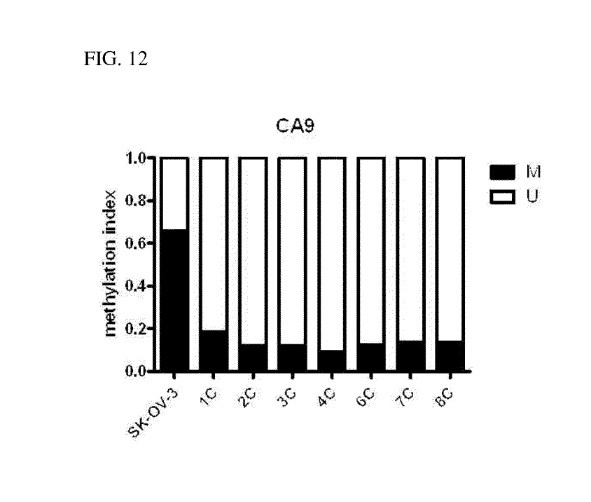

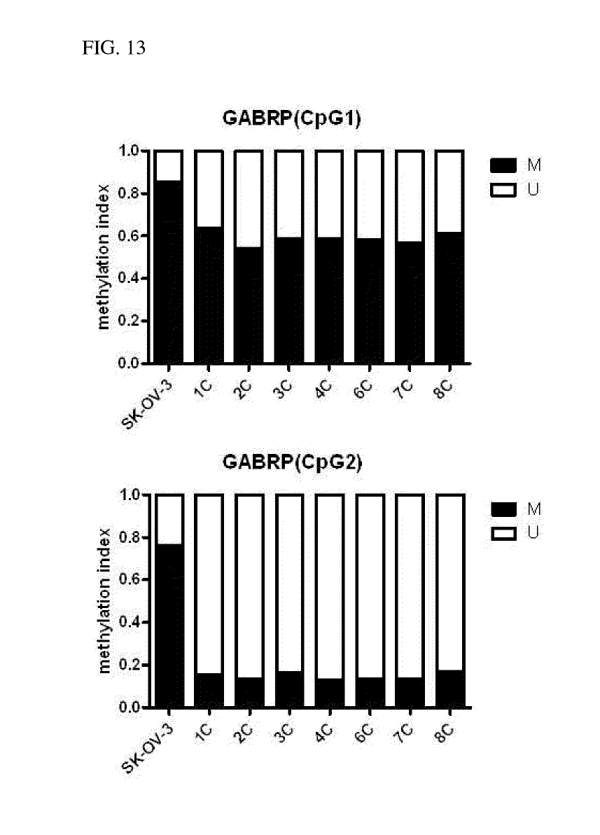

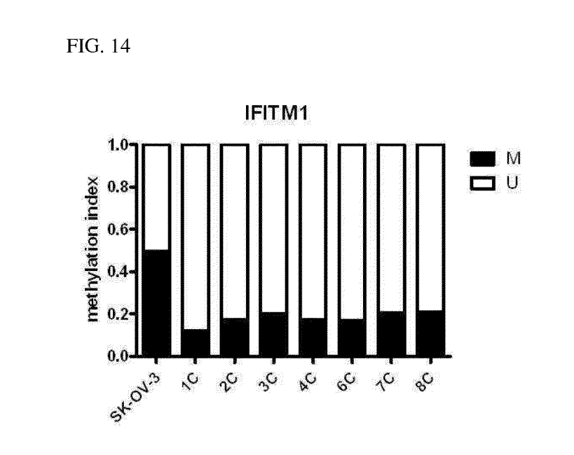

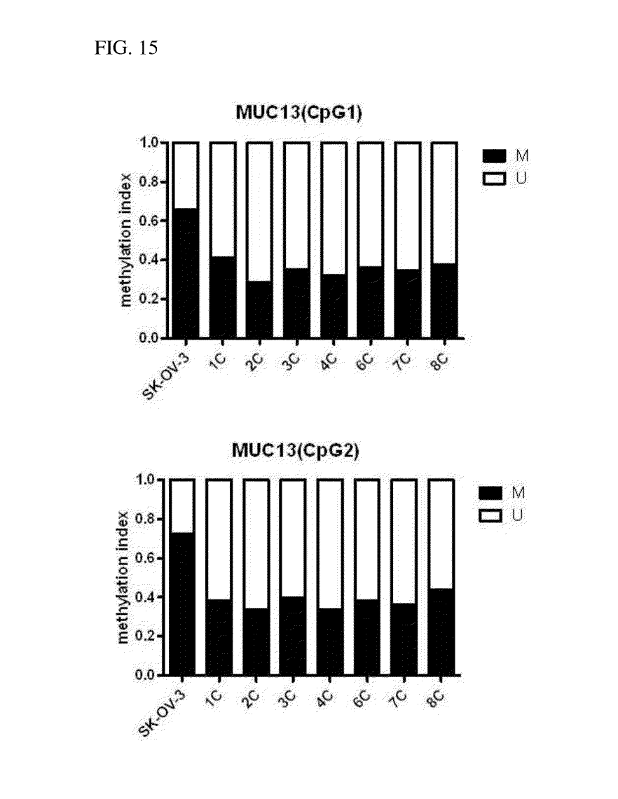

Further, the result of analyzing the DNA methylation microarray showed a remarkable reduction in DNA methylation at the specific CpG site (ch7: 16844546-16844667) in the promoter of AGR2 gene. Specific hypomethylations were observed at the specific CpG site (ch9: 35673849-35673970) in the promoter of CA9 gene, at the specific CpG sites (ch5: 170209700-170209821 and ch5: 170209521-170209642) in the promoter of GABRP gene, at the specific CpG site (ch11: 313984-314105) in the promoter of IFITM1 gene, and at the specific CpG sites (ch3: 124653658-124653779 and ch3: 124653599-124653720) in the promoter of MUC13 gene (FIGS. 11 to 15).

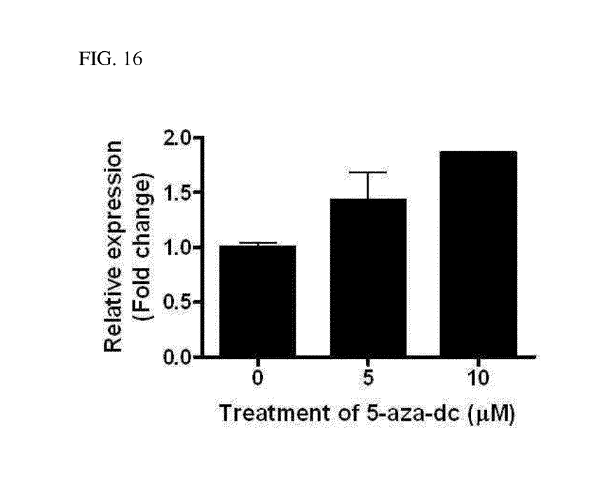

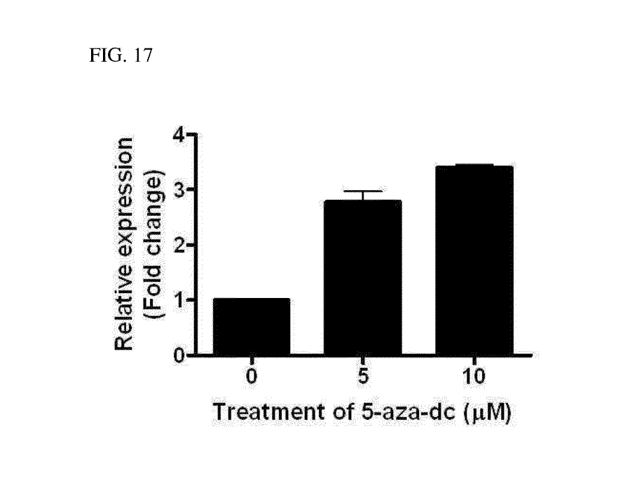

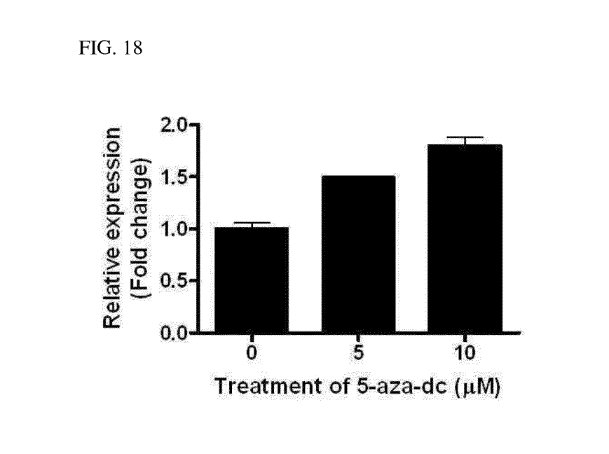

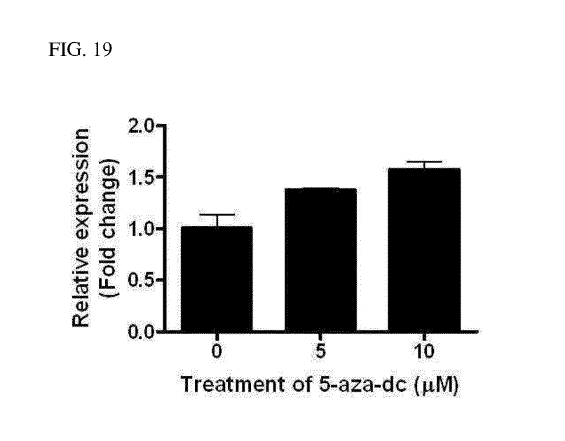

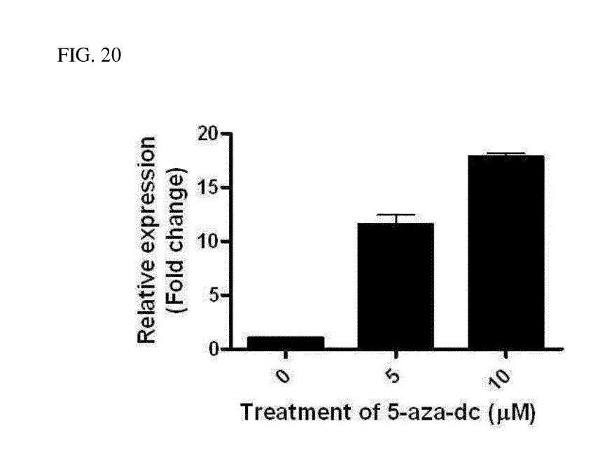

Further, changes in expressions of the five genes (AGR2, CA9, GABRP, IFITM1, and MUC13) were examined after treatment of the primary cell line SK-OV-3 with the demethylating agent, 5-aza-2′-deoxycytidine for 3 days. As a result, increased expressions of the above genes were observed with reduced DNA methylation, indicating that the expressions of the above five genes are regulated by DNA methylation (FIGS. 16 to 20).

These experimental results showed that the abrupt increase in the five genes (AGR2, CA9, GABRP, IFITM1, and MUC13) in the ovarian cancer metastasis model is regulated by hypomethylation at the specific CpG site of the promoter of each gene, which is an ovarian cancer metastasis model-specific phenomenon.

What is claimed is:

1. A method for diagnosing ovarian cancer metastasis or the risk of the metastasis, comprising the steps of:
(a) measuring methylation levels at the CpG sites of one or more gene promoters selected from the group consisting of AGR2 (anterior gradient 2), CA9 (carbonic adj anhydrase 9), GABRP (gamma-aminobutyric acid receptor pi subunit), IFITM1 (interferon-induced transmembrane 1) and MUC13 (mucin 13) in a biological sample of a subject,
(b) comparing the methylation levels with those of the gene promoters of a control sample, and
(c) determining that the subject has ovarian cancer metastasis or is at the risk of the metastasis when the methylation levels measured in the sample of the subject are lower than those of the control sample.

2. The method according to claim 1, wherein the step (a) is performed by using a compound modifying an unmethylated cytosine base or a methylation sensitive restriction enzyme, primers specific to the methylated sequence of the gene promoter, and primers specific to the unmethylated sequence.

3. The method according to claim 2, wherein the step (a) includes the steps of treating the genomic DNA obtained from the sample with the compound modifying an unmethylated cytosine base or the methylation sensitive restriction enzyme; and measuring the methylation level of the treated DNA by one or more methods selected from the group consisting of methylation-specific polymerase chain reaction, real time methylation-specific polymerase chain reaction, PCR using a methylated DNA-specific binding protein, quantitative PCR, pyrosequencing and bisulfite sequencing using primers capable of amplifying the methylated region of the gene promoter.

4. The method according to claim 2, wherein the compound modifying an unmethylated cytosine base is bisulfite or a salt thereof.

5. The method according to claim 2, wherein the methylation sensitive restriction enzyme is SmaI, SacII, EagI, HpaII, MspI, BssHII, BstUI, NotI.

6. The method according to claim 1, wherein the CpG site of the AGR2 gene promoter includes CpG in the base sequence of SEQ ID NO. 1.

7. The method according to claim 1, wherein the CpG site of the CA9 gene promoter includes CpG in the base sequence of SEQ ID NO. 2.

8. The method according to claim 1, wherein the CpG site of the GABRP gene promoter includes CpG in the base sequence of SEQ ID NO. 3 or SEQ ID NO. 4.

9. The method according to claim 1, wherein the CpG site of the IFITM1 gene promoter includes CpG in the base sequence of SEQ ID NO. 5.

10. The method according to claim 1, wherein the CpG site of the MUC13 gene promoter includes CpG in the base sequence of SEQ ID NO. 6 or SEQ ID NO. 7.


SEQUENCE LISTING (19 sequences)

SEQ ID NO: 1 - DNA, 122 bp, Homo sapiens
  promoter (1)..(122): CpG region in the promoter of AGR2 (the base 16844546 to 16844667 of chromosome 7)
tcaggagccttacctggatttcctcacccacctgccttgtgtgagtcggcggctaggatgcggtccaagcttctgagtgtgccagcacagctgagtctctatttatgcaccagggcatacct

SEQ ID NO: 2 - DNA, 122 bp, Homo sapiens
  promoter (1)..(122): CpG region in the promoter of CA9 (the base 35673849 to 35673970 of chromosome 9)
gagtcagcctgctcccctccaggcttgctcctcccccacccagctctcgtttccaatgcacgtacagcccgtacacaccgtgtgctgggacaccccacagtcagccgcatggctcccctgtg

SEQ ID NO: 3 - DNA, 122 bp, Homo sapiens
  promoter (1)..(122): CpG region in the promoter of GABRP (the base 170209700 to 170209821 of chromosome 5)
gctccccttcctttctctctctcccttcccggctcctagactgggtctaggcaggagcttcgccatgatgtcaggagggaaggagatgcctctgggcctcgtaccacagcctcagctctctc

SEQ ID NO: 4 - DNA, 122 bp, Homo sapiens
  promoter (1)..(122): CpG region in the promoter of GABRP (the base 170209521 to 170209642 of chromosome 5)
gagtcctctcaggagaaatgaaagcgctgtctctttaggtgcccactccttacccaccagcggagcaatgttttccaggctggccaggctccagaagctcactaaggaagttggcttggggc

SEQ ID NO: 5 - DNA, 122 bp, Homo sapiens
  promoter (1)..(122): CpG region in the promoter of IFITM1 (the base 313984 to 314105 of chromosome 11)
aatttacaaacagcaggaaatagaaacttaagagaaatacacacttctgagaaactgaaacgacaggggaaaggaggtctcactgagcaccgtcccagcatccggacaccacagcggccctt

SEQ ID NO: 6 - DNA, 122 bp, Homo sapiens
  promoter (1)..(122): CpG region in the promoter of MUC13 (the base 124653658 to 124653779 of chromosome 3)
acgtcaaaccacagaaaggcaattatagctagcatttggccaaataccatagatctcttccgagcttttgagatagaagaggtggagctggccttacgaatcatttttctgactcaaagccc

SEQ ID NO: 7 - DNA, 122 bp, Homo sapiens
  promoter (1)..(122): CpG region in the promoter of MUC13 (the base 124653599 to 124653720 of chromosome 3)
ccttcctggctacctttcgttttaactgtctttcgggtacaaagtccagtcattaattgacgtcaaaccacagaaaggcaattatagctagcatttggccaaataccatagatctcttccga

SEQ ID NO: 8 - DNA, 24 bp, Artificial Sequence
  human AGR2 primer (forward)
agtttgtcctcctcaatctggttt

SEQ ID NO: 9 - DNA, 23 bp, Artificial Sequence
  human AGR2 primer (reverse)
gacatactggccatcaggagaaa

SEQ ID NO: 10 - DNA, 23 bp, Artificial Sequence
  human CA9 primer (forward)
tgactctcggctacagctgaact

SEQ ID NO: 11 - DNA, 19 bp, Artificial Sequence
  human CA9 primer (reverse)
ccactccagcagggaagga

SEQ ID NO: 12 - DNA, 21 bp, Artificial Sequence
  human GABRP primer (forward)
ctcgattcagtccctgcaaga

SEQ ID NO: 13 - DNA, 17 bp, Artificial Sequence
  human GABRP primer (reverse)
gtgcgggacccgatcat

SEQ ID NO: 14 - DNA, 20 bp, Artificial Sequence
  human IFITM1 primer (forward)
cgccaagtgcctgaacatct

SEQ ID NO: 15 - DNA, 25 bp, Artificial Sequence
  human IFITM1 primer (reverse)
taccagtaacaggatgaatccaatg

SEQ ID NO: 16 - DNA, 23 bp, Artificial Sequence
  human MUC13 primer (forward)
agaaacattccatggcctatcaa

SEQ ID NO: 17 - DNA, 23 bp, Artificial Sequence
  human MUC13 primer reverse)
tgtccataaacagatgtgccaaa

SEQ ID NO: 18 - DNA, 20 bp, Artificial Sequence
  human 18S rRNA primer (forward)
cggctaccacatccaaggaa

SEQ ID NO: 19 - DNA, 18 bp, Artificial Sequence
  human 18S rRNA primer (reverse)
gctggaattaccgcggct